(19) United States
(12) Patent Application Publication
(10) Pub. No.: US 2024/0191183 A1
(43) Pub. Date: Jun. 13, 2024

(54) DESTRUCTIBLE MICROWELL ARRAYS FOR PARTICLE SEPARATION AND ANALYSIS

(71) Applicant: ENRICH BIOSYSTEMS INC., Branford, CT (US)

(72) Inventor: Qi ZHAO, West Hartford, CT (US)

(21) Appl. No.: 18/285,723

(22) PCT Filed: Apr. 29, 2022
(86) PCT No.: PCT/US2022/026955

§ 371 Date: Oct. 5, 2023

Related U.S. Application Data
(60) Provisional application No. 63/182,972, filed on May 2, 2021.

(51) Int. Cl. C12N 5/00

(52) U.S. Cl. CPC C12N 5/0068; C12N 2533/72; C12N 2533/74; C12N 2533/76; C12N 2533/78

(57) ABSTRACT

The present invention provides destructible, digestible, or dissolvable microwell arrays, such as hydrogel microwell arrays, which are useful for segregating, culturing, and analyzing biological samples such as cells and mixtures of cells.

### CROSS-REFERENCE TO RELATED APPLICATION

This application is a national phase entry under 35 U.S.C. § 371 of International Application PCT/US2022/026955, filed Apr. 29, 2022, which claims priority to U.S. Provisional Application No. 63/182,972, filed May 2, 2021, each of which are hereby incorporated by reference in their entirety herein.

### FIELD OF THE INVENTION

The present invention provides destructible, digestible, degradable, or dissolvable microwell arrays (also abbreviated as “DMWs”), such as hydrogel microwell arrays, which are useful for segregating, culturing, and analyzing cell samples. Also provided are essential device, software, and protocols to generate and use these microwell arrays.

### BACKGROUND OF THE INVENTION

Primary tumor derived cells, including tumor cells, lymphocytes and healthy cells can provide valuable insights into disease etiology and treatments. These cell collections are generally heterogeneous, and the insights are useful for basic research as well as applied precision medicine and basic research.

In the United States alone, it is believed that more than 8000 NIH funded research projects are currently investigating primary tumor derived cells for basic research and clinical studies, i.e., pharmacological screening, neoantigen discovery and cell therapy etc. Single cell tools enabling individual clone tracking and culturing are superior in understanding disease progression, maintaining clone diversity, sample representativeness and clinical relevance. However, bulk culturing methods still dominate ex-vivo expansion for its simplicity and flexibility. There is a need for sufficiently easy methods to enable high-diversity clonal growth and tracking while still supporting the invested bulk culture protocols, imaging instruments and downstream steps.

Isolating particles and cells directly under the microscope is still a challenge in the biomedical field. Using microscopy, it is relatively easy to identify cells of specific morphological phenotypes or those interacting with other cell types. However, it is much more difficult to select cells or particles of interest from a sample without employing submicron-precision mechanical instruments and methods (e.g., micro-pipetting, optical tweezers, or laser micro-dissection). The major current competitive technologies used to isolate particles or cells are flow-cytometry, microfluidic devices, micro-pipettes, and optical tweezers.

The challenge of isolating, identifying, and quantifying cells is even more difficult when the objective is to perform the methodology on a heterogeneous cell sample population. Ex vivo expanded heterogeneous cell populations offer the potential for direct characterization of e.g., disease biology, drug response, and sample genotyping, and hence has been gaining momentum in different biological fields. Ex vivo expansion refers to culturing a sample such as a cell, tissue, or tumor sample to expand, increase, or amplify the amount of sample. However, two characteristics of heterogeneous cell populations, restrict the wide application of routine characterization methods, namely the loss of clone diversity and an often limited time-window in which to perform the analyses. Furthermore, selection-absence or selection-presence of ex-vivo expansion of a heterogeneous cell population can result in growth bias, which can limit the value of the expanded sample such that the expanded sample is no longer representative of the cell population diversity of the original sample. The reason for this is that not all cells in a heterogeneous population grow and replicate at the same rate. For example, cells in a heterogeneous sample that have a more rapid replication rate and/or that are more viable can overtake slower-replicating and/or less viable cells in the sample. Thus, a heterogeneous cell sample that is cultured for replication and then characterization and quantitation, can be very different after culturing compared to the initial cell sample, and would thus not be representative of that initial cell sample. In other words, the culturing and amplification can add unwanted bias into the methodology such that the resultant cell culture is no longer representative of the original cell culture. As such, the ex vivo expanded culture may not have the intended utility, such as for example the preparation of a tumor biopsy sample for evaluation against potential chemotherapeutic agents.

Adding to these challenges is that current methods for isolating and segregating cell types from a heterogeneous cell sample are difficult or impractical, if not downright impossible. As mentioned above, techniques based on microscopy, cytometry, and micro-tweezers, and suction isolation devices are not applicable, as these are generally directed at isolating single cells or small groups of cells from simpler sample systems, and are not intended for or practical for large complex heterogeneous cell samples. Also, flow-cytometry set-ups can be expensive and require significant laboratory space, and as just stated are not amenable for the isolation of single cells.

Live single cell isolation technologies have potential value on multiple fronts of life-science research, such as antibody development, primary cell separation, cell line construction, immune cell therapy, and circulating tumor cell (CTC) separation. An advantage of the present invention is a that it provides a low-cost, robust mechanism which enables live cell isolation under challenging scenarios: such as for limited sample sizes, clumpy samples, and adherent cells. The present invention addresses many of the shortcomings of current technologies for segregating cells from samples such as heterogeneous biological samples for characterization and quantitation.

The present invention provides systems and methods to convert traditional plates for culturing cells into a single cell imaging/culturing platform which can provide a cell diversity of greater >10⁵. The systems and methods of the present invention can be useful, for example, for phenotypically profiling tumor cell pools. It is believed the systems and methods can be useful for the preservation of phenotypical/genetic heterogeneity. The resultant high diversity cell cultures obtained can be used for cell subtype specific pharmacological screening and for the preparation of tumor specific T cell based therapies.

The destructible microwell systems and methods of the present invention address the shortcomings and disadvantages of currently available systems and methods and provide for the generation of ex vivo sample cultures that are representative of the original sample and that can be used, for example, for research purposes, drug evaluation and development, and personalized medicine. In particular the present invention provides a means for developing targeted cancer therapies for patients.

Furthermore, biological methods using ex vivo expanded heterogeneous cell populations can offer direct insights into disease biology and drug response. Such, ex vivo methods are gaining momentum in many different fields. However, in practice, the lack of simplicity and flexibility has hindered existing tools for tackling tumor heterogeneity.

It is apparent from the foregoing that there is an ongoing need for developing devices, systems, and methodologies for segregating, culturing, and analyzing cell heterogeneous cell samples. The present invention addresses these needs.

### SUMMARY OF THE INVENTION

The present invention provides destructible, digestible, degradable, or dissolvable microwell arrays, such as hydrogel microwell arrays, which are useful for segregating, culturing, and analyzing cell samples.

In an embodiment, the present invention also provides essential device, software, and protocols to generate and use these microwell arrays.

In an embodiment, the present invention provides a method for separation and analysis of biological particles utilizing a destructible hydrogel microwell comprising the steps of:
a) establishment of an array of hydrogel microwells,
b) seeding a sample of biological particles into the array,
c) culturing (or incubating) the sample, and
d) destruction of the microwells to release the cultured biological particles from the microwells.

In further embodiments, the culturing step c) produces cultured biological particles within the microwells.

In further embodiments, the method comprises a further step, e) of quantitating and/or identifying the released particles from the destroyed microwells.

In further embodiments, the method comprises the further step x) between step c) and step d) of
x) segregation of targeted microwells from the array.

In further embodiments, the hydrogel is optically transparent.

In further embodiments, the hydrogel is transparent to light from about 315 nm to about 400 nm.

In further embodiments, each hydrogel microwell of the array has a diameter, width, or cross-sectional dimension from about 1 micron to 10 mm.

In further embodiments, the depth (inside height of the walls) of each hydrogel microwell of the array is from about 10 microns to about 500 microns.

In further embodiments, the volume of each hydrogel microwell of the array is from about 1×10⁻¹² liters to about 1×10⁻⁶ liters.

In further embodiments, the microwell array comprises from about 2 to about 1×10¹⁰ microwells.

In further embodiments, the microwell array comprises from about 1×10³ to about 1×10⁸ microwells.

In further embodiments, the microwell hydrogel array is a 2D array.

In further embodiments the microwell hydrogel array is a 3D array.

In further embodiments, the hydrogel is selected from the group consisting of gelatin and its derivatives, agarose and its derivatives, dextran and its derivatives, cellulose and its derivatives, chitin and its derivatives, alginate and its derivatives, PEG and its derivatives, and combinations thereof.

In further embodiments the hydrogel is established by a photo-initiator.

In further embodiments the photo-initiator is lithium phenyl(2,4,6-trimethylbenzoyl)phosphinate.

In further embodiments, the hydrogel array is bound to or capable of adhering to a substrate.

In further embodiments, the substrate is selected from the group consisting of polystyrene, polyacrylate, polycarbonate, co-polymers of polystyrene, polyacrylate, and/or polycarbonate, and glass.

In further embodiments, the destruction of step d) is selected from the group consisting of partial destruction and complete destruction.

In further embodiments, the destruction of step d) is performed by a method selected from the group consisting of,
i. chemical means (including enzymatic means),
ii. light means (including UV and visible),
iii. thermal means,
iv. sonic means (applying sound energy),
v. physical means,
vi. electromagnetic radiation,
vii. atomic particle means,
viii. subatomic particle means,
ix. biological means (degradable by cells or tissues),
and combinations thereof.

In further embodiments the destruction of step d) is performed by enzymatic digestion.

In further embodiments, the enzymatic digestion if performed with an enzyme selected from the group consisting of collagenase, trypsin, cellulose hydrolase, alginate lyase, dextranase, accutase, and combinations thereof.

In further embodiments, the enzymatic digestion is performed in the presence of EDTA or EGTA (also known as CAS 67-42-5 or ethyleneglycol-bis(β-aminoethyl)-N,N,N′,N′-tetraacetic Acid).

In further embodiments, the biological particles are cells.

In further embodiments, the cells are selected from the group consisting of tumor cells, healthy cells, mutated cells, T-cells, lymphocytes, stem cells, circulating tumor cells, virus infected cells, adherent cells, suspension cells, and combinations thereof.

In further embodiments, the cells are selected from the group consisting of bacteria, plants, fungi, and combinations thereof.

In further embodiments, the cells are further carrying nucleic acid fragments (such as DNA fragments and/or RNA fragments), mutations in their genomes, plasmids, or wherein the cells are part of a microbiome containing a variety of microorganism species.

In further embodiments, the quantitation and/or qualitative analysis step e) is performed by morphology, kinetics, growth curve, cell killing, cell surface marker, migration, interaction, genome sequencing, fluorescence, illuminance, reporter gene expression, transcriptome sequencing, mass-spectrum, secreted proteins, and imaging.

In an embodiment, the present invention provides for a device (hardware and software) for generating a hydrogel microwell array having one or more of the characteristics disclosed herein.

In an embodiment, the present invention provides for destructible hydrogel microwell array construct having one or more of the characteristics disclosed herein.

In an embodiment, the present invention provides for a method for preparing a destructible hydrogel microwell array comprising the steps of:
(a) depositing a polymerizable hydrogel onto a substrate, and
(b) initiating polymerization of the hydrogel with an energy source projected onto the hydrogel in a predetermined microarray arrangement to generate the microarray.

In further embodiments, the substrate is selected from the group consisting of polystyrene, polyacrylate, polycarbonate, co-polymers of polystyrene, polyacrylate, and/or polycarbonate, and glass.

In further embodiments, the deposition step (a) further comprises depositing a photo-initiator and a light absorption, and wherein in step (b) the energy source is a light source.

In further embodiments, the polymerizable hydrogel is selected from the group consisting of gelatin-methyl acrylate, dextran-methyl acrylate, and combinations thereof; the photoinitiator is lithium phenyl(2,4,6-trimethylbenzoyl)phosphinate; and the light absorption agent is tartrazine.

In further embodiments, the light source is a widefield (4 mm in diameter), narrow angle 405 nm laser.

In further embodiments, the laser is projected onto a 2K TFT (thin film transistor) monochrome LCD display to generate the microwell array.

In various further embodiments, quantitation and/or qualitative analysis step e) (of quantitating and/or identifying the released particles from the destroyed microwells) can be used for the following:
(i) T-cell isolation
(ii) for cancer diagnosis or prescription of a treatment regimen based on tumor typing. For example, high diversity tumor infiltration lymphocyte generation. The cytotoxic T-cells in tumor infiltrating lymphocytes (TIL) have conferred anti-tumor activity in multiple clinical trials, where ex-vivo expansion of TIL is generally needed to achieve the therapeutic dose. Clonal growth bias remodels TCR repertoire (17), which implied poor representation of tumoricidal clones and reduced coverage of tumor mutations (18). For tumor infiltrated lymphocytes (TIL), the preservation of genomic diversity is in principle necessary to cover the tumor mutation pool, then the ex-vivo expanded TIL can be collected for cell therapy.
(iii) Preservation of tumor heterogeneity during patient derived tumor cell line culture. 1: Loss of cancer cells due to stroma outgrowth. Primary tumor samples are known to be burdened with stroma cells; the outgrowth of such non-tumor cells are the top reasons accounted for tumor cell line failures. It has been shown in a 538-case study designed to address primary tumor cell line challenges showed that the overall success rate of establishing tumor cell lines from primary tumors is only ˜9%-38%, varying significantly among different tumor types (15). Optimizing medium components have been the normal strategy to selectively suppress fibroblasts outgrown, which requires individual effort on a case-by-case basis. 2: Loss of rare tumor subtypes. Owing to different developmental stages of tumorigenesis, subtypes of tumor cells display varied degree of metastasis tendency, differentiation potency and growth rate (16). Simultaneous tracking of starting rare clonotypes are highly desirable for either understanding tumor evolution or for pharmacological screening. Especially for pharmaceutical screening, if specific sub-populations of tumor cells, genetically and phenotypically, can be specifically monitored and targeted, a combination treatment are likely to be more successful than monotherapies. 3: Loss of tumor mutations. Primary tumor cells harbor the 100-10000 (1) somatic mutations deep genotyping of these mutations is the basis for neoantigen discovery. In-vitro expansion of primary tumor cells almost always results in distorted mutation pool. Therefore, large mass of tumor samples is used in place of prolonged cell culture to ensure sampling relevance, which hinders its application using micro-biopsies. Using DMW, such losses of heterogeneity will be minimized, and
(iv) Coculture, monitor and clonally expand immune cells and tumor cells interaction. Heterogenous immune cells and tumor cells can be mixed and seeded into such hydrogel microwells, the tumor killing clones of immune cells can be visualized and clonally expanded with or without external stimulations (i.e. cytokine stimulation) within individual microwells. such clones can later be pooled or isolated after destruction of hydrogel microwell clones. Immune cells can be native cells or genetically modified cells; they can be B-cells, nature killer cell, T-cells and other tumor suppression cells.

These and other aspects of the present invention will become apparent from the disclosure herein.

### BRIEF DESCRIPTION OF THE DRAWINGS

FIG. 1 shows the dissolvable microwell culture workflow and microwell generation technology. FIG. 1A top row shows the schematics of the workflow operation with sample loading, cell culture/analysis and cell retrieval. FIG. 1A bottom row shows the corresponding scenarios in the petri dish with the square in the circle representing the destructible microwell array in a petri dish and the ovals representing cells. FIG. 1B is an image of top and front views of a destructible microwell generating machine, where a 35 mm-petri dish is loaded via an adapter. The platform is also compatible with most off-the-shelf microtiter plates, when adapter is not needed. FIG. 1C is an image of a 1.4K microwell system (on a 10 mm diameter region-petri-dish). In the embodiment, up to 12K microwell can be generated on a 28 mm diameter region-petri-dish.

FIG. 2 shows fast microwell generation and reagent optimization. FIG. 2A is a schematic view of a liquid crystal display (LCD) masked microwell generation. FIG. 2B is a typical LCD pattern for generating a 250 micron microwell system. FIG. 2C is a real time photo from the microwell generating process where a 405 nm light spot is visible (indicated by arrow). FIG. 2D shows a series of 8 microwell arrays generated using different hydrogel materials for evaluating optical transparency and structure integrity, which can be visually checked. The third microwell array from left in the top row (outlined) is a gelatin-methyl acrylate based microwell, which has optimized properties.

FIG. 3 shows holding suspension cells using the microwells of the present invention microwells. FIG. 3A is a schematic of particle trapping using the microwells. FIG. 3B shows time lapsed photos of fast division of suspension cells over a period of 48 hours. The dimension of the microwell is about 350 microns and the nucleus is stained with a red live cell tracking dye. Photos were taken every 15 minutes over the 48 period, but only 8 photos are shown for simplicity at approximate times points of 0 hours, 5 hours, 10 hours, 20 hours, 28 hours, 35 hours, 42 hours, and 48 hours.

FIG. 4 demonstrates facile cell retrieval by enzymatic digestion of the microwell array. FIG. 4A is an overview of hydrogel based microwells of different dimensions. FIG. 4B, left panel shows a hydrogel microwell after 5 min of collagenase II digestion (0.1 μ/ml at 37° C.), where there has been partial microwell digestion. Note the remaining microwell structures still remaining. Approximately 5 Jurkat cells are present in each well. FIG. 4B, right panel shows a hydrogel microwell after 15 min into digestion, where the wall structures are completely dissolved, and cells are left intact for retrieval.

FIG. 5 shows localized clonal expansion of adherent tumor cell pools. 5K of C4-2 cells were seeded into one well of a 24 well culture plate with and 4K microwell. A set of overlapped brightfield and mCherry channel images were collected daily, full well montages were synthesized and two corresponding sections at day 1 (top panel) and day 8 (bottom panel) are shown.

FIG. 6 shows a Model system of preferred tumor growth via stroma inhibition. Tumor (monoclonal C4-2 mCherry) and single layer forming, endothelial cell line (EAHY) are mixed at 1:3 ratio. 5K of cells from the same mixture were seeded into two wells of 24 well culture plate with (FIGS. 6A and 6B) and without (FIGS. 6C and 6D) 4K microwell. After 14 days of coculture, a set of overlapping images covering entire well were collected and collaged. The overall fluorescent views (FIG. 6A and FIG. 6C) and close up views (FIG. 6B and FIG. 6D) of two juxtaposed samples are shown for comparison.

FIG. 7 shows a overview schematic of phenotypical profiling of tumor cell pools in an elongated hydrogel array. Elongated microwells are shown as rectangles. Individual clone images taken at time zero (TO) are shown with dashed circumferences, and are shown at 48 hours (T1) as solid line ovals.

FIG. 8 shows the clonal expansion of Jurkat cells in DMW with a typical clone image at day 1 and after seeding at day 3, day 6, day 7 and day 8.

FIG. 9 shows the clonal expansion of Jurkat cells in DMW as a normalized growth curves per microwell for a 255 well 2-dimensional array.

### DETAILED DESCRIPTION OF THE INVENTION

### Definitions

As used herein, the following terms and abbreviations have the indicated meanings unless expressly stated to the contrary.

The abbreviation “DMW” means destructible microwell. Alternatively, the abbreviation can mean digestible microwell, degradable microwell, or dissolvable microwell.

The term “ex-vivo expansion” as used herein means growing or culturing cells outside of a living organism under artificial conditions to increase or amplify the sample.

The term “hydrogel” as used herein means a crosslinked hydrophilic polymer.

The abbreviation “LCD” as used herein means liquid-crystal display.

The term “microwell” as used herein refers to a very small well, receptacle, or container. The microwells of the present invention are composed of a hydrogel material. These hydrogel microwells are generally produced or arranged in a 2-dimensional (2-D) or 3-dimensional (3-D) array of a multitude of individual microwells.

### Systems of the Present Invention

The present invention comprises one or more destructible, digestible, degradable, or dissolvable hydrogel microwells, preferably arranged in an array of two or more microwells, and more typically in an array of as many or more of 10,000 microwells.

Ex vivo expanded heterogeneous cell populations can offer direct characterization of disease biology, drug response and sample genotyping and hence has been gaining momentum in many different fields. These fields include basic research, drug development and evaluation, and personalized or precision medicine. An example of such personalized or precision medicine is the culturing of tumor biopsies from cancer patients for the characterization of the tumor for the 25 development of a targeted treatment regimen for the patient. Nevertheless, two particularities apparently obstruct its wide application of ex vivo expansion, namely the loss of clone diversity and limited time-window in which to culture the cells. In principle, any selection-absence or selection-presence of the ex-vivo expansion of a heterogenous cell population could result in growth bias of the sample, which could limit its utility. Therefore, current ex vivo expansion methods could inevitably benefit from the destructive microwell technology of the present invention.

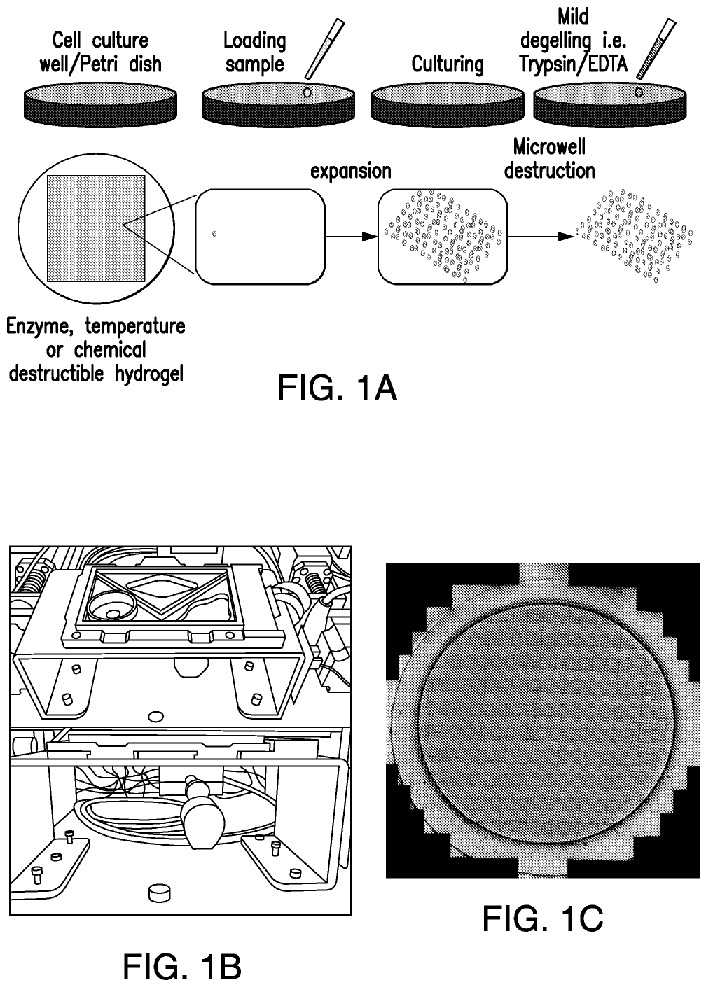

In related fields such as genomics, the preservation of clone diversity is attempted by droplet or microwell technology, where in both cases, single cells or doublet cells are compartmented and the growth limit of each clone is restrained by the physical space of the droplet or micro-well. Droplets require mechanical or fluidic operations to disperse the cells into the droplet and a tedious process of in-drop culturing and droplet breaking, and hence droplet technology is not suitable for many cell manipulation requirements. On the other hand, ex vivo expansion methods employing conventional microwells hinder the facile cell retrieval. The destructible microwell systems and methods of the present invention obviate these shortcomings. The basic concept of the destructible microwell cultures of the present invention is set forth in FIG. 1.

The described technology includes establishment of hydrogel microwells, seeding of an initial cell sample pool, culturing the cells in the microwells, and cell retrieval. Cell retrieval is achieved using enzymatic, light induced, radiation induced, pH induced, temperature induced, or chemical induced-destruction of the microwells. Either complete or partial destruction of microwells can be performed. See FIG. 1. Furthermore, a segregation step can be performed to selectively separate microwells of interest. The microwells can be established in an array of about 10³ to about 10¹⁰ microwells. Individual microwells can have a diameter, width, or horizontal/cross-sectional dimension (length or width) on the order from about 1 micron to about 10 mm. Individual microwells have a depth (inside height of the walls) of about 10 microns to about 500 microns, with about 100 microns being a convenient depth. Volumes of the microwells can range from about 1×10⁻¹² liters to about 1×10⁻⁶ liters. The microwells can be either uniform or not (e.g., symmetric or asymmetric), and can be established in a 2-dimensional (2-D) or 3-dimensional (3-D) array. The given microwell dimensions and volumes are exemplary and it can be appreciated that microwell dimensions and volumes outside of these ranges are contemplated in alternative embodiments. It can also be appreciated that a person of skill in the art can choose microwell dimensions to achieve a target microwell volume, or alternatively can choose a microwell volume to achieve one or more target microwell dimensions.

The described technology, though prominently useful for selection-free cell expansion, and should be considered superior in diversity preservation in all cases where cellular diversity is of concern. The materials of the microwells include those such that the microwells have sufficient strength and integrity for the cell culture aspects and any segregation procedures, but that can be destroyed, digested, or dissolved when needed and under appropriate controlled conditions. The microwells can be made from materials including, but not limited to, gelatin and its derivatives, agarose and its derivative, dextran and its derivatives, chitin and its derivatives, alginate and its derivatives, PEG (polyethylene glycol) and its derivatives, PPG (polypropylene glycol) and its derivatives, mixed PEG/PPG polymers (mixed polyethylene glycol and polypropylene glycol), cellulose and its derivatives, etc. The present invention demonstrates the feasibility and practicality of ex-vivo cell expansion using such microwells, with a further controlled destruction to release the contents for harvesting and further use and characterization.

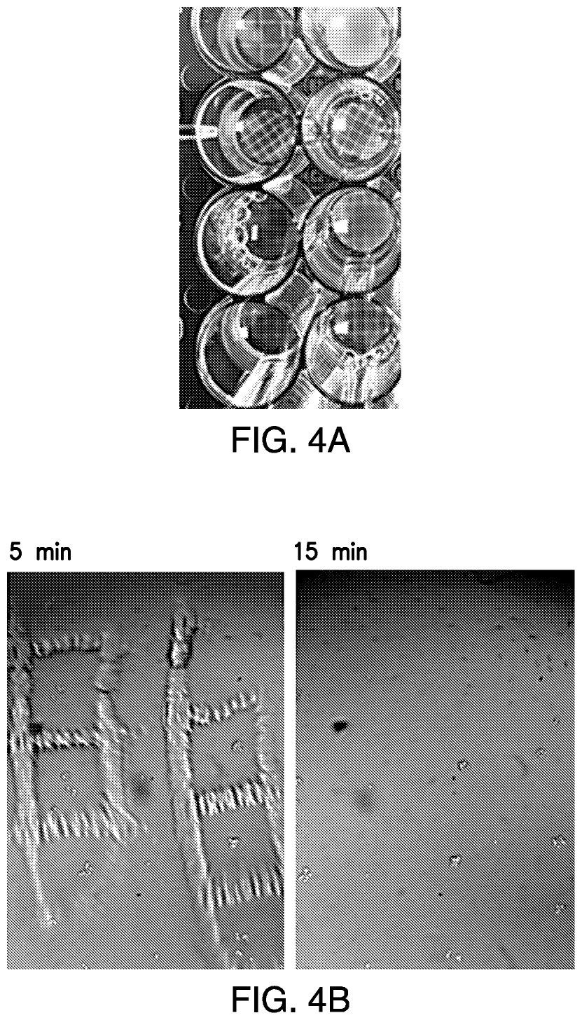

As stated above, ex vivo expanded heterogeneous cell populations offer direct characterization of disease biology, drug response and sample genotype, hence has been gaining momentum in many different fields. However, conventional methods suffer from loss of clone diversity and limited time-window of use. Therefore, a diversity-retaining expansion method without the requirement for extra and time-consuming manipulation would be highly desirable for such applications. We here provide a destructible microwell system and method. For example, we show a cell diversity of and have achieved retrieval using collagenase digestion. See FIG. 4A.

As an example of the challenges which the present invention addresses, in regard to tumor infiltration lymphocytes (TIL) therapies (i.e., the generation of anti-tumor clones by cell expansion), assuming a 10³-10⁴ initial retrieval of TILs from a 1-3 mm³ tumor incision, up to a 10 million-fold (15-20 generations) of clone expansion would have to be achieved to provide a useful sample. Considering this clone expansion, the slightest growth advantages among lymphocytes clones would be amplified and reflected as a significant and undesirable growth bias. Such bias can entail the following injurious consequences: (i) Growth-advantaged clones, which are normally not tumor specific clones, would dominate and dilute the efficacious population, so much so that high quantities of cells required to achieve therapeutical thresholds in TIL treatment would be diminished. (ii) Tumor tissue normally harbors heterogeneous mutations, whereas reduced T cell diversity could implicate incomplete response or a recurrence post TIL infusion therapy. (iii) In cases with fewer initial reactive TILs, such as “cold” tumors, growth bias stemming from for example peripheral blood mononuclear cells (PBMC) alone could eliminate anti-tumor clones by competition in cytokine, nutrition and space, as is consistent with the modest success that can be achieved in non-melanoma TIL trials. Furthermore, selection pressures have been introduced to promote on-target proliferation hence preserve useful clones. In achieving this, extra immunological or cytometry steps are needed. These steps in turn prolong and complicate TIL production, requiring longer preparation processes which often yield unfavorable cellular characteristics, e.g., shortened telomeres and loss of activating receptors. With respect to TIL quality, minimal cell manipulation equals population diversity in importance.

The systems and methods of the present invention can be used to segregate a cell type of interest from a larger cell sample mixture. For example, the systems and methods can be used to remove a certain cell strain from a complex mixture such as in microbiome applications where a particular bacterium might be deleterious and one might aim to remove it while maintaining the diversity of the remainder of the sample. Also, another use would be to segregate a particular cell tumor mutation from a larger collection of cells from a tumor sample. Yet a further use would be to remove undesired cells from a sample while maintaining the remaining describable cells.

We here provide a destructible (e.g., in some embodiments an enzymatically digestible) hydrogel based microwell technology that requires no additional step or instrumentation in preparing the ex vivo TIL culture, where the cell clones can be easily distributed to the bottom of each micro well. At the end of the culture period, the expanded cells can be easily retrieved by a brief collagenase incubation. Our technology can increase the active population of TILs, reduce preparation time and the quantity requirement of the final TIL product, and expand the scope of TIL therapy to other tumors. In addition, the present invention does not require specific cell markers for labeling nor cell-cell separation. We can validate this technology using PBMC expansion and tumor cell culturing. The expansion rate and genomic diversity can be compared to bulk culturing and is applicable to other cell population cultures.

### Hydrogels

As stated above, hydrogels are crosslinked hydrophilic polymers. Hydrogels are generally highly absorbent, do not dissolve in water, and maintain a well-defined structure. Hydrogels can be both naturally derived or synthetically made. Hydrogels can also be of the chemical type or physical type. Chemical hydrogels have covalent cross-linking bonds, whereas physical hydrogels have non-covalent bonds, such as hydrogen bonds.

Hydrogels are prepared using a variety of polymeric materials. These materials can be divided broadly into two categories according to their origin: natural or synthetic polymers. Natural polymers for hydrogel preparation include hyaluronic acid, chitosan, heparin, alginate, agarose, cellulose, methyl cellulose, peptides, and fibrin. See, Kharkar, Prathamesh M.; Kiick, Kristi L.; Kloxin, April M. (5 Aug. 2013). “Designing degradable hydrogels for orthogonal control of cell microenvironments”. Chemical Society Reviews. 42 (17): 7335-7372. doi:10.1039/C3CS60040H. PMC 3762890. PMID 23609001. Common synthetic polymers include polyvinyl alcohol, polyethylene glycol, polypropylene glycol, sodium polyacrylate, acrylate polymers, methacrylate polymers, and copolymers thereof. See, Cai, Wensheng; Gupta, Ram B. (2012). “Hydrogels”. Kirk-Othmer Encyclopedia of Chemical Technology. doi:10.1002/0471238961.0825041807211620.a01.pub2. ISBN 978-0471238966.

Many of the naturally derived hydrogels can have desirable biodegradability qualities, giving rise to their use herein for preparing destructible microwell arrays of the present invention.

Nonlimiting examples of hydrogels useful herein include those selected from the group consisting of gelatin and its derivatives, agarose and its derivative, dextran and its derivatives, chitin and its derivatives, alginate and its derivatives, PEG and its derivatives, PPG and its derivatives, cellulose and its derivatives, agarose and its derivatives, and combinations thereof. Common derivatives include acrylate and methacrylate (methyl acrylate) derivatives. The hydrogels can also comprise a combination of the materials described in the prior sentence within a single polymeric structure. In other words, the hydrogel polymer is a copolymer of these materials.

The hydrogels useful herein are easily destructible, digestible, degradable, or dissolvable so that the contents of the microwells constructed from the hydrogels can be isolated, collected, and analyzed. The destruction of the microwells can be performed by means selected from the group consisting of:
i. chemical means (including enzymatic means),
ii. light means (including UV and visible light)
iii. thermal means
iv. sonic means (e.g., sonication)
v. physical means (e.g., cutting, shearing, mixing, homogenizing)
vi. electromagnetic radiation,
vii. atomic particle means,
viii. subatomic particle means,
ix. biological means (degradable by cells or tissues),
and combinations thereof.

The hydrogels and their arrays should be optically transparent, which means that they allow for the passage of visible or visible and ultraviolet radiation. The wavelength of optical transparency should be from about 380 nm to about 740 nm which are considered the wavelength for visible light and from about 315 nm to about 380 nm. It is recognized that these wavelength ranges are approximate because different scientific sources recite slightly different ranges.

### Destructible, Digestible, Degradable, or Dissolvable Microwells

The term “microwell” as used herein refers to a very small well, receptacle, or container composed of a hydrogel material of the present invention. The microwells of the present invention are generally produced or arranged in a 2-dimensional or 3-dimensional array of a multitude of individual microwells. These microwell arrays are useful for the present methods for containing the biological cell samples for culturing, segregation, isolation, and analysis.

The hydrogel microwells are consequently readily destructible, digestible, degradable, or dissolvable, by the means described herein for the particular hydrogel material.

The microwells can be established in from about 10³ to about 10¹⁰ scale. Each microwell can have a dimension or diameter from about 1 micron (μ) to about 10 mm. The microwells can be either uniform or not, and can be established in both 2-dimensional or 3-dimensional arrangements.

The described technology, though would be prominently useful for selection-free cell expansion, and should be considered useful for diversity preservation where preserving the cellular diversity of a sample is of concern.

### Microwells, Systems, and Methods of the Present Invention

The present invention provides destructible, i.e., digestible, degradable, or dissolvable, microwell (DMW) technology, whereby as many as tens of thousands of microwells can be easily generated and ready to use in less than about an hour, and typically within about 20 minutes. Using these microwells, single clones of heterogenous cells can be segregated, individually tracked, and analyzed using pre-established protocols for bulk culturing. In some instances, using enzymatic digestion, the cells can be easily retrieved for a variety of downstream utilities, i.e., FACS, (fluorescence-activated cell sorting), proteomics, sequencing or as cell based therapeutics at the end of the expansion. The technology is expected to be of relatively lost cost and efficiency and can be customizable by the user. The systems and methods of the present invention can generate, for example an array of up to about 10,000 or more microwells.

However, there have been challenges with existing micro-compartmentalization technologies for primary tumor derived cells Clone diversity has been well recognized in the genomics field. Encapsulation technologies such as emulsion/droplet (2) and plastic/glass chips (2-5) are used to restrain each clone or DNA to a defined physical space, which has demonstrated some success in removing bias during bulk amplification processes such as PCR (polymerase chain reaction). However, existing encapsulation technologies usually do not suffice in simplicity and flexibility to be of practical daily use for researchers and medical diagnostic applications.

The challenges with current technologies can be generally broken down into three areas: (i) lack of compatibility with existing bulk culture protocols, (ii) difficulties with cell retrieval, and (iii) lack of flexibility.

Regarding the lack of compatibility with existing bulk culture protocols, living cells are sensitive. Culture conditions and imaging platforms have been heavily optimized in bulk format. Specifically, droplet/emulsion-based technologies (6-9) tend to be better for throughput and retrieval. However, it is difficult to track individual clones, so that clone behaviors such as cell fate and migration cannot be readily observed. Furthermore, the different aeration/nutrition conditions between encapsulated droplets and bulk cultures often requires extensive adaption efforts from bulk protocols.

Regarding the difficulties with cell retrieval, microwells are more similar to bulk cultures and compatible with existing analysis tool. Without specialized equipment, cells are difficult to retrieve from pre-made glass/plastic microwells, preventing the adoption of such microwells into existing pipelines utilizing subsequent FACS, proteomics, and sequencing analysis, or as a drug modality. These “hard” wells are also expensive to manufacture and customize.

Regarding the lack of flexibility, hydrogel microwells have been reported by individual labs, mostly requiring fixed wafer based photo-masks (10-14). The ability of customizing the shape and dimensions are important for researchers for different purposes. For example, small wells can be more appropriate for short-term single cell morphology analysis, whereas larger wells can be more appropriate for long term growth studies, and elongated wells can be more appropriate for cell mobility measurements.

A drawback of bulk cell culturing is the loss of tumor heterogeneity. When cultured in bulk, the uneven growth rate of individual clones in tumor derived cell pools has led to loss of tumor heterogeneities, thereby creating inconsistencies and failures.

Conventional bulk culturing can lead to the loss of cancer cells due to stroma outgrowth in these cultures. Primary tumor samples are known to be burdened with stroma cells; the outgrowth of such non-tumor cells are among the top reasons accounted for tumor cell line failures. A 538-case study designed to address primary tumor cell line challenges showed that the overall success rate of establishing tumor cell lines from primary tumors is only ˜9%-38%, varying significantly among different tumor types (15). Optimizing culture medium components has been the normal strategy for selectively suppressing fibroblast outgrowth, which requires individual effort on a case-by-case basis.

Conventional bulk culturing can also lead to the loss of rare tumor subtypes. Owing to different developmental stages of tumorigenesis, subtypes of tumor cells display varied degrees of metastasis tendency, differentiation potency and growth rate (16). Simultaneous tracking of starting rare clonotypes are highly desirable for either understanding tumor evolution or for pharmacological screening. This tracking is especially important for pharmaceutical screening (both genetically and phenotypically), if specific sub-populations of tumor cells can be specifically monitored and targeted. In such instances, appropriate combination treatments are more likely to be successful than monotherapies.

Conventional bulk culturing can also lead to the loss of tumor mutations. Primary tumor cells generally harbor as many as 100-10,000 somatic mutations. (1) Deep genotyping of these mutations is the basis for neoantigen discovery. In-vitro expansion of primary tumor cells very often results in distorted mutation pools. Therefore, large masses of tumor samples are generally used in place of prolonged cell culturing to ensure sampling relevance. The requirement for large tumor mass samples hinders applications using micro-biopsies.

Conventional bulk culturing can also lead to the loss of tumor infiltrating lymphocyte (TIL) clones. The cytotoxic T cells in tumor infiltrating lymphocytes (TIL) have been shown to confer anti-tumor activity in multiple clinical trials, where ex-vivo expansion of TIL is generally needed to achieve a therapeutic dose. Clonal growth bias remodels T-cell receptors (TCR) (17), which implies poor representation of tumoricidal clones and reduced coverage of tumor mutations (18).

Therefore, a high-diversity, clone-trackable cell culture platform that still supports existing cell biology protocols, i.e., drop-in seeding, pipetting media exchange and enzymatic cell retrieval, is needed for interrogating heterogeneous tumor samples among larger research communities. The destructible microwell technology of the present invention fits this need.

The approach of the present invention involves several facets including devices, software, and protocols to generate the 10,000+ diversity destructible microwells. The technology of the present invention is applicable for durability and compatibility with traditional culture protocols using tumor cells which are adherent or in suspension.

The present invention should have applicability for promotion of primary tumor cell outgrowth in the presence of stroma contamination, examples of which could entail the following: phenotypical profiling of mouse tumor derived cell pools, to support clone-trackable pharmacological screening, tumor specific lymphocyte expansion, investigation of tumor killing activities, and of expanded tumor-infiltrating lymphocytes (TIL).

### Examples

The following examples further describe and demonstrate embodiments within the scope of the present invention. The Examples are given solely for purpose of illustration and are not to be construed as limitations of the present invention, as many variations thereof are possible without departing from the spirit and scope of the invention.

### 1. System Assembly and Operational Validation of Destructible Microwell Enabled Cell Cultures.

Operational feasibility, event density, optical transparency, and maintaining cell functions are the popular features of traditional petri-dish based bulk cell cultures, which need to be preserved during microwell formation. For seamlessly adapting the destructible microwell technology into a well-established cell culturing-analysis pipeline, a sufficiently simple and fast method to generate and dissolve microwells is also desirable or required for practical applications. Mechanically, the hydrogel based well system, though soft in nature, should be microscopically rigid for segregating cells, surviving disturbances, and restraining mobile cells. Additionally, for clone tracking, an automatic image stitching and microwell image segmentation software is an additional feature that can be employed.

### 2. Software and Hardware of a Hydrogel Microwell Generation Device.

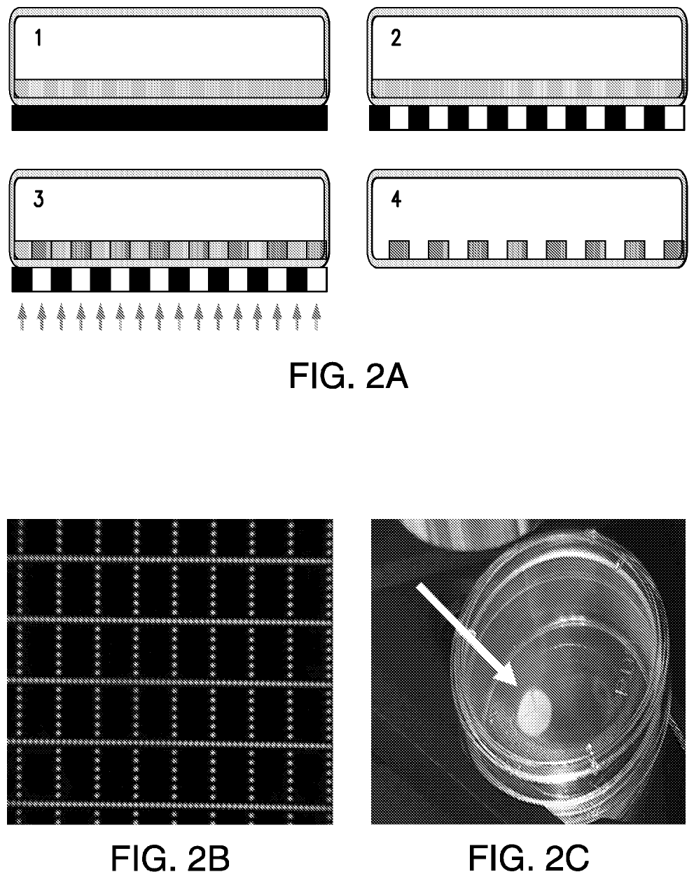

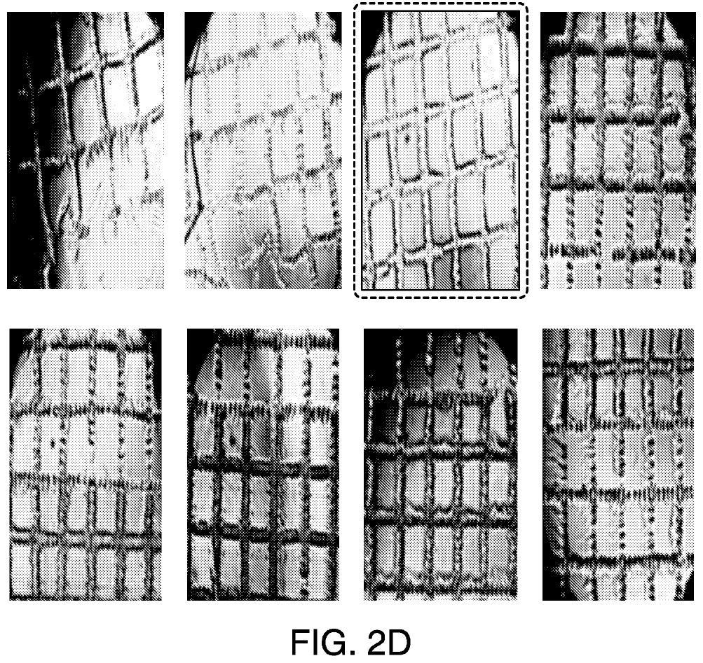

The destructible microwells are generated by hydrogel lithography, where the sizes and shapes of the microwell can be designed by the software (FIG. 2A) and projected onto a 2K TFT (thin film transistor) monochrome LCD display (FIG. 2B). A widefield (4 mm in diameter), narrow angle 405 nm laser is used to induce the gelation of various photopolymerization hydrogels (FIG. 2C), most of which are enzymatically digestible. Using gelatin-methyl acrylate and dextran-methyl acrylate, we have ascertained desirable compositions of hydrogels which can be optimized to form optically transparent grid systems that bind well to polystyrene or glass-based Petri dish/microtiter plates (FIG. 2D), by screening hydrogel material, the selected concentration of gel-methyl acrylate and a photo-initiator [lithium phenyl(2,4,6-trimethylbenzoyl)phosphinate, which is also known as LAP, a photo-curable bioink] and a light absorption agent (tartrazine). The microwell generation software can be fully parameterized. The generating scanning speed can be around 20 mm²/min with the resulting wall of ˜80 microns in height. The petri-dish of 10K microwells can be automatically generated within about 30 minutes and subsequently rinsed with phosphate-buffered saline (PBS) for cell culture use. Minimal hands-on time is 30) involved.

Hydrogel lithography techniques may generally follow steps 1˜4 depicted in FIG. 2A. In step 1, the petri dish or suitable containment vessel (i.e. a substrate) is filled with a hydrogel forming solution while the dish or vessel sits on top of an LCD. Alternatively, the dish or vessel may be filled with the hydrogel forming solution and then placed on an LCD. In step 2, a grid pattern on the LCD is turned on to allow light to pass at specific positions to define the grid pattern. The positions at which light is allowed to pass will define the walls of the microwell. In step 3, a curing light source is provided to induce hydrogel polymerization at the positions of the grid where light is allowed to pass through the LCD. In step 4, the un-solidified hydrogel forming solution is washed away to leave the microwell pattern. It can be appreciated that any combination of steps or alternative methods are contemplated for producing the microwells of the present disclosure.

For clone tracking by registering each microwell, we provide software to take in a collage of overlapping images covering the entire destructible microwell system. Such image datasets can be collected from a motorized microscope or the optical module of the destructible microwell system generating system of the present invention. A whole well montage can be generated and each microwell can be picked out, indexed, and tracked. 15

### 3. Micro-Trapping and Content Holding Capacity of Suspension Cells Over a Period of Time.

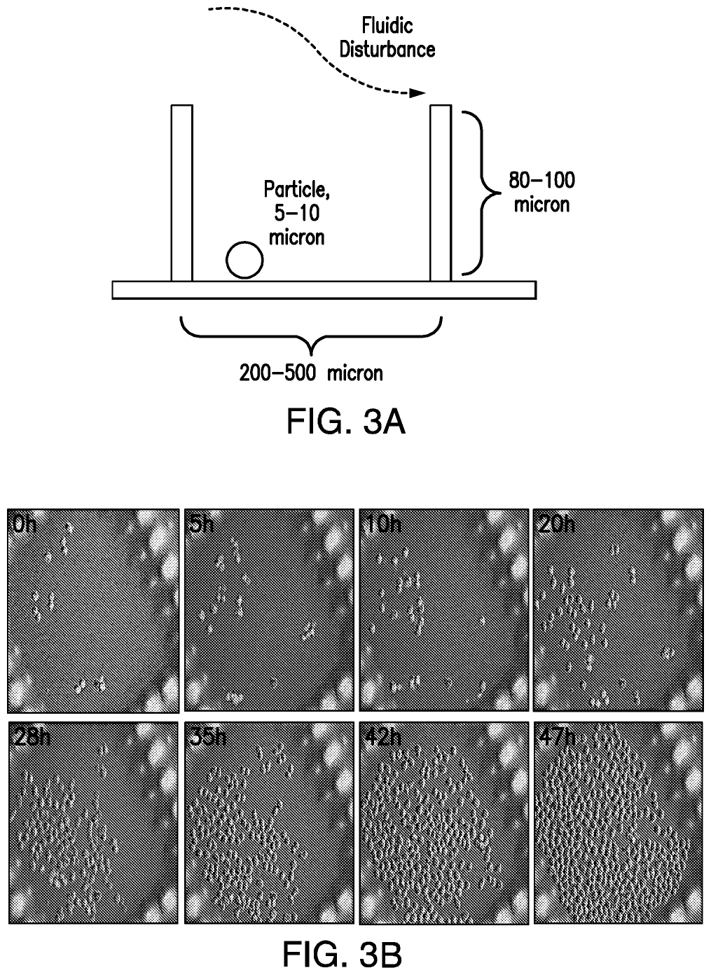

Owing to the dimensional difference between fluidic movement and the size of the microwell, particles that have settled at the bottom of microwell are generally not affected by macroscopic disturbances. The micro-trapping property of the destructible microwell system can be used to segregate each clone, whether adherent or suspensive, into a high well-density system, and to register individual clone behavior by microwell indexing. Also, such trapping capacity can be resistant to common cell culture operations such as medium changing and plate loading for imaging, thereby allowing the use of an off-the-shelf cell culture container as a single cell visualization tool, without the need for modifying established protocols. For long term clone holding and for restraining mobile cells, the present invention demonstrates cell restraining capacity against a group of rapidly growing and moving primary cells (FIG. 3). During a period of 48 hours and longer, we have not observed detectable cell evasion from the microwell.

### 4. Reagents for Microwell Dissolution.

The microfluidic trapping effect of the microwells provides resistance against the cells being undesirably washed out, which had previously rendered prior microwell devices as a terminal, analysis-only tool, and not for cell culture and amplification purposes. Compared with glass/plastic microwells, the present invention is superior for cell retrieval. Because the hydrogel material is enzymatically digestible, the entire well structure can be completely liquified for downstream work such as flow cytometry, protein/DNA extraction, and therapeutic modalities. For gelatin-methyl acrylate based microwells, we show that the destructible microwells can be efficiently digested within minutes (FIG. 4B).

### 5. Single Clone Segregation Efficiency

For cell seeding, cells are resuspended in the culture medium, and applied directly onto the destructible microwells through random deposition. Cells normally settle within about 1 hour. With a given cell/well ratio, the cell number in individual wells follows Poisson statistics. The single clone ratio can be readily tuned between 0.2 to 0.8 by tuning the well/cell ratio from 1:1 to 1:10.

### 6. Considerations for Performance

For robust performance of destructible microwells for optimal clarity and dissolution of the material, consideration can be given to compositions such as the following hydrogel/hydrogel-hydrolase pairs including: gelatin-methyl acrylate/collagenase, dextran-methyl acrylate/dextranase, chitin-methyl acrylate/chitinase, and alginate-methyl acrylate/lyase. The wall height of the destructible microwells can be customized in response to parameters of different light doses and gel composition, where the height can be measured by being pushed under the microscope. Typical heights, essentially the depths of the microwells, are about 10 microns to about 100 microns, with 80 microns being convenient

### 7. Adherent Cells: Expansion, Phenotypical and Genetic Diversity of Solid Tumor Cells.

Cells tend to migrate and aggregate in bulk culture, making individual clones difficult, if not impossible, to be tracked over a period of time. The direct utility of the present invention is the clone-wise phenotypical profiling of a heterogenous pool of cells including growth, differentiation, migration, and key cell marker expression. Moreover, the physical segregation of individual clones can lead to contact inhibition for normal cells and the outgrowth of tumor cells, which can greatly improve the success rate of making primary tumor cell lines. The mutation pool of tumor cell pools propagated in the microwells can be compared with original sample to evaluate the ability of the technology to maintain the original mutations.

### 8. Clonal Growth of Adherent Tumor Cell Line—Experimental Design and Outcomes.

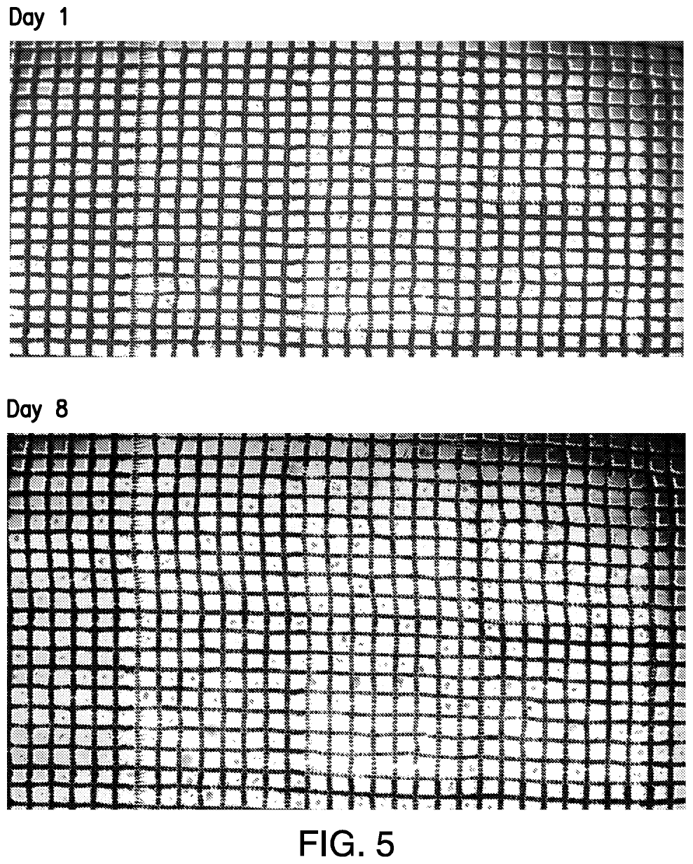

To ascertain whether the growth rate of adherent tumor cells is comparable with bulk culturing and whether the hydrogel generated wall can efficiently provide a barrier between different adherent cells, we used an mCherry transfected epithelial cancer cell line C4-2 pool as a model system to culture in a 24 well microtiter plate with and without 4000 microwells in identical media [DMEM (Dulbecco's Modified Eagle Medium) medium, 5% FBS (fetal bovine serum), 0.1% Pen/Strep (penicillin-streptomycin)]. During the prolonged culturing we observed a similar growth speed. Unlike a huge clump in bulk cultures, cells in the destructible microwells formed relatively uniform cell aggregates (FIG. 5), with no crossover events being observed.

### 9. Limiting Stroma Outgrowth Using a Co-Culture Model System—Experimental Design and Outcomes

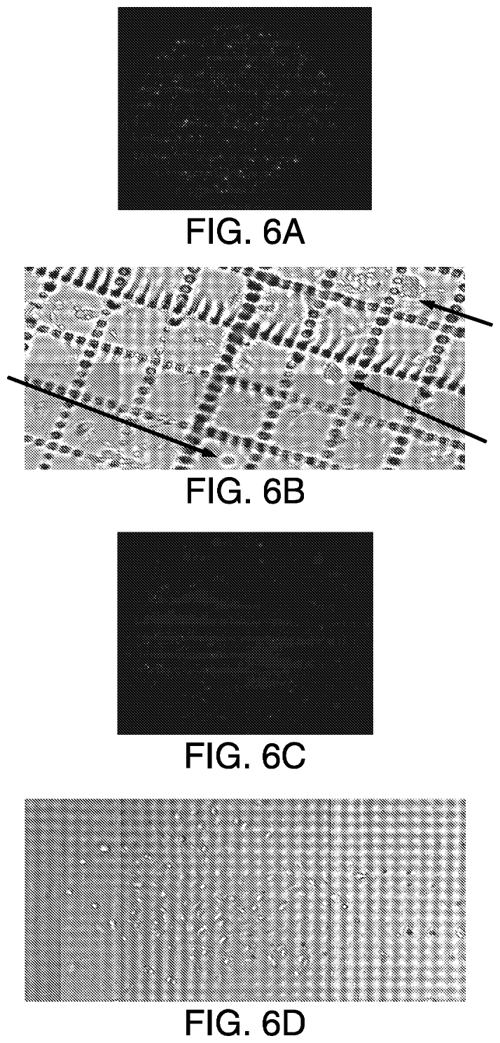

To model the cogrowth of tumor and normal tissue fibroblasts, we used a mixture of prostate tumor derived C4-2 cells and a single layer forming endothelial cell line (EAHY) at a ratio of 1:3. We observed self-limiting growth after single layer formation within the microwells, while tumor cells such as C4-2 are not limited by contact. After 14 days of coculture, the full-well fluorescent view showed a more abundant mCherry signal in the destructible microwell sample than from a traditional bulk sample. Detailed views showed consistent results demonstrating that without the competition of stroma cells, tumor cells are capable of forming spherulites (FIG. 6). Additional ratios using different tumor types and primary fibroblast cells can be evaluated. The end coculture can be retrieved by collagenase digestion and the elevation of tumor abundance with/without the destructible microwells can be quantitatively studied. Consistent tumor outgrowth observed among most tumor types suggests a high success rate for constructing primary tumor derived cell lines.

### 10. Expansion and Behavior Profiling of Heterogenous Cell Pools in an Elongated Destructible Microwell Array

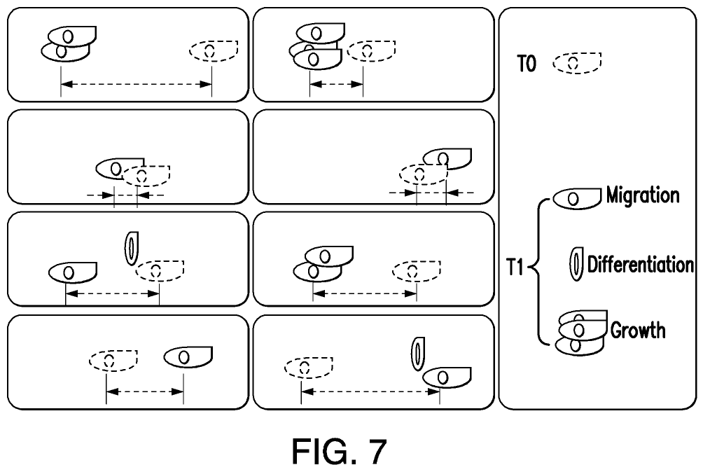

The destructible microwell technology enables direct utilization of existing culture protocols and analysis equipment, which has been developed in conventional bulk cell cultures. Microwell shapes can also be customized using destructible microwell technology. Elongated microwells can be generated for quantification of cell migration (FIG. 7). Mouse tumor and matching tissues are purchased from Jackson laboratories. The majority of the material is reserved as a benchmark, while 10⁵ tumor derived cells can be cultured in the microwells and in bulk. We then establish a framework of multiparameter profiling of tumor pool characterizing clone behaviors including migration, differentiation, expansion rate, and essential markers such as CD133 expression by live time imaging, using the microwells. Live cell imaging can be used to help quantify the cell behaviors and cellular markers. The dataset is analyzed and visualized using dimension reduction methods. Using this dataset, we can cluster the entire cellular pool into quiescent, spherulite forming, migrating, and candidate tumor stem species, as the foundation of subgroup specific pharmacological screening.

### 11. Sequencing and Evaluating Tumor Specific Mutations.

We validated whether destructible microwells can protect the intactness of a tumor mutation pool after expansion, using samples retrieved from an enzymatic hydrogel, the RNA of which is reverse transcribed into a cDNA library. The quality of total RNAs extracted from the sample can be evaluated with TapeStation2200 (Agilent Technologies). A 5′ rapid amplification of cDNA end adapter can be added during the cDNA synthesis using a SMART cDNA library construction kit (Clontech). 25 The transcriptome of the bulk culture sample, original sample and matching normal tissue can be sequenced using MiSeq, mapped to a mouse reference proteome. Tumor specific non-silent mutations can be recorded together with the individual abundance. The mutation spectrums of the destructible microwell culture samples can be compared with the bulk culture and original samples. The top mutations with over 1% abundance identified in the original sample can be counted in the two expanded samples. Loss of significant mutations or gain of de novo mutations can be counted as a deviation from the original sample. A cross correlation can be calculated to quantify the pool similarity.

### 12. Statistical Considerations

For statistical significance, culturing assays can be performed in triplicate with averages and standardized variations of migration distance, growth rate, and mutation abundances calculated for comparison. Consideration of balanced representation in gender, age: tumor sections, and TIL samples can be sourced from mice of different gender and ages.

### 13: Suspensive Cells: Stimulated Expansion and Genetic Diversity of Tumor Infiltrating Lymphocytes

For tumor infiltrated lymphocytes (TIL), the preservation of genomic diversity is in principle necessary to cover the tumor mutation pool. Moreover, ex-vivo expanded TILs are generally required for cell therapy. The combination of the two requirements makes it a useful application of the destructible microwell technology. We can use mouse TIL to demonstrate and evaluate the conditions of expanding TIL in vitro, characterizing the growth curve, evaluating optimal expansion protocols, and the upkeep of T-cell receptor genetic diversity.

### 14. Observation and Quantifying Single Cell Growth Curves Using a Spiked in Cell Line.

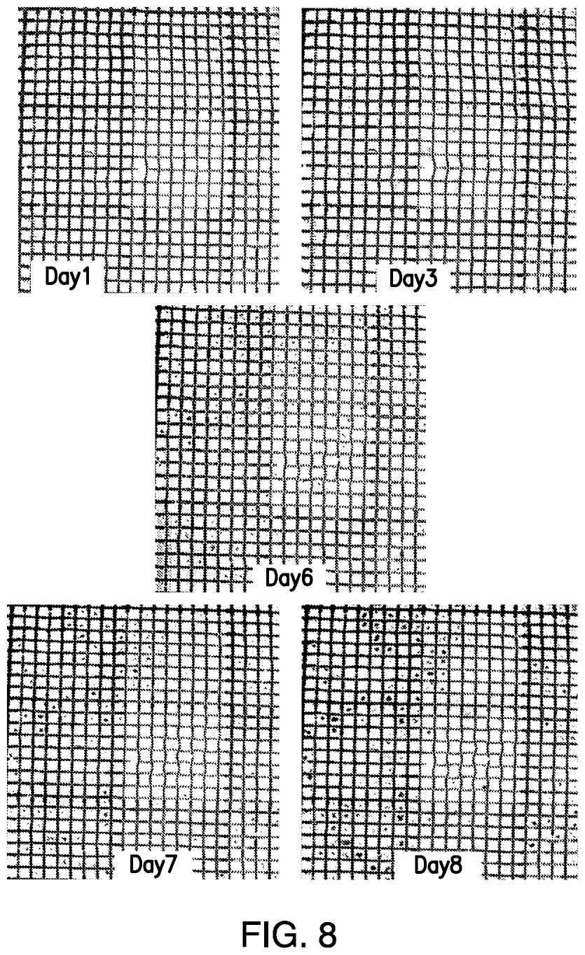

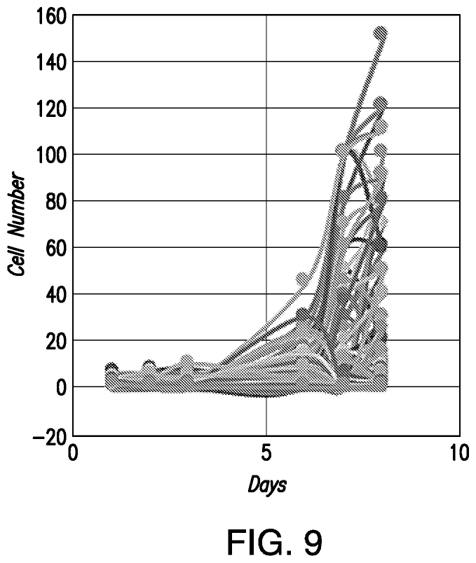

One concern of micro-compartmented cell cultures is that the less mobile cultures in microwells conditions might negatively affect clone division, which is essential to match disease progression in the clinical setting. We validated the long-term suspension culture capacity of the destructible microwell technology using Jurkat cells. 2K cells were seeded into a 4K destructible microwell cultured with RPMI1640 10% FBS (fetal bovine serum). Whole well images at different time points were collected and collaged. Individual microwell images were collected and cell numbers were counted using particle recognition algorithms. Individual growth curves were plotted. We observed a typical exponential growth similar to that for a bulk culture (FIGS. 8 and 9-10).

### 15. Interleukin-2 Stimulated Mouse TIL Expansion in Microwells—Reverse Cell Marker Labeling Using Clone Tracking.

It is of high value to optimize different cytokines to specifically expand certain subtypes of lymphocytes in research and in therapy. Nevertheless, immune labeling of primary cells can interfere with such process. We can take advantage of the bird's-eye view of individual clones and the retrospective tracking of clone growth to achieve a tempering-free method to evaluate different stimulation methods on the subsets of the TILs. We can use different concentration of IL2 (interleukin-2) to parallel destructible microwell cultured TIL pools. The image of each well can be recorded and the clone specific growth curve can be calculated. At the end of the expansion, we can apply ant-CD4 and anti-CD8 antibodies to the samples. The identity of cells in each microwell is then determined and the growth rates of CD4+ T cells and CD8+ T cells grouped and analyzed against different concentration of IL2.

16. T-Cell cDNA Library Generation and T-Cell Receptor (TCR) Diversity Comparison

cDNA libraries are generated as described above in Example 11 using a SMART cDNA library construction kit (Clontech). T-cell receptor-β sequences can be amplified using a forward primer for the SMART adapter and a reverse primer specific to the TC-cell receptor constant region. An Illumina sequence adapter with barcode sequences is then added. The final prepared libraries can be sequenced by MiSeq (Illumina Inc.). The CDR3 (third complementary determining region of the heavy chain) can be translated and extracted using read alignment to T-cell receptor (TCR) reference sequences obtained from IMGT/GENE-DB (http://www.imgt.org) using Bowtie2 (19) aligner. TCR-β3 diversity can be calculated and compared among original TIL, bulk culture and destructible microwell culture using TCR (20).

Non-single clone microwells can be removed during analysis. For statistical significance, we can perform all culturing assays in triplicate, averages and standardized variations of grow rate, and TCR abundances can be calculated for comparison. For cell loading consistency, cell mixtures can be passed though through 30-micron cell mesh before the seeding step. In addition to top abundant clone distributions, for more comprehensive comparisons, total TCR diversities from different culturing methods can be quantified using multiple statistical methods such as Shannon entropy (21), Gini-Simpson index (22) and evenness measures such Pielou's index (23).

### REFERENCES

1. McGranahan N, Swanton C. Clonal Heterogeneity and Tumor Evolution: Past, Present, and the Future. Cell. 2017; 168 (4):613-28.
2. Aigrain L, Gu Y, Quail M A. Quantitation of next generation sequencing library preparation protocol efficiencies using droplet digital PCR assays—a systematic comparison of DNA library preparation kits for Illumina sequencing. BMC Genomics. 2016; 17:458.
3. Clark I C, Delley C L, Sun C, Thakur R, Stott S L, Thaploo S, et al. Targeted Single-Cell RNA and DNA Sequencing With Fluorescence-Activated Droplet Merger. Anal Chem. 2020; 92 (21):14616-23.
4. Rhee M, Light Y K, Meagher R J, Singh A K. Digital Droplet Multiple Displacement Amplification (ddMDA) for Whole Genome Sequencing of Limited DNA Samples. PLOS One. 2016; 11 (5):e0153699.
5. Snyder R P, Guerin M T, Hargis B M, Imai R, Kruth P S, Page G, et al.

Exploiting digital droplet PCR and Next Generation Sequencing technologies to determine the relative abundance of individual Eimeria species in a DNA sample. Vet Parasitol. 2021; 296:109443.
6. Bjork S M, Sjostrom S L, Andersson-Svahn H, Joensson H N. Metabolite profiling of microfluidic cell culture conditions for droplet based screening. Biomicrofluidics. 2015; 9 (4):044128.
7. Huang H, Yu Y, Hu Y, He X, Berk Usta O, Yarmush M L. Generation and manipulation of hydrogel microcapsules by droplet-based microfluidics for mammalian cell culture. Lab Chip. 2017; 17 (11):1913-32.
8. Kim T, Cho Y H. A pumpless cell culture chip with the constant medium perfusion-rate maintained by balanced droplet dispensing. Lab Chip. 2011; 11 (10):1825-30.
9. Zhao S P, Ma Y, Lou Q, Zhu H, Yang B, Fang Q. Three-Dimensional Cell Culture and Drug Testing in a Microfluidic Sidewall-Attached Droplet Array. Anal Chem. 2017; 89 (19):10153-7.
10. Barrett T A, Wu A, Zhang H, Levy M S, Lye G J. Microwell engineering characterization for mammalian cell culture process development. Biotechnol Bioeng. 2010; 105 (2):260-75.
11. Bernard A B, Lin C C, Anseth K S. A microwell cell culture platform for the aggregation of pancreatic beta-cells. Tissue Eng Part C Methods. 2012; 18 (8):583-92.
12. Kobel S A, Lutolf M P. Fabrication of PEG hydrogel microwell arrays for high-throughput single stem cell culture and analysis. Methods Mol Biol. 2012; 811:101-12.
13. Moeller H C, Mian M K, Shrivastava S, Chung B G, Khademhosseini A. A microwell array system for stem cell culture. Biomaterials. 2008; 29 (6):752-63.
14. Randall C L, Kalinin Y V, Jamal M, Manohar T, Gracias D H. Three-dimensional microwell arrays for cell culture. Lab Chip. 2011; 11 (1): 127-31.
15. Kodack D P, Farago A F, Dastur A, Held M A, Dardaei L, Friboulet L, et al. Primary Patient-Derived Cancer Cells and Their Potential for Personalized Cancer Patient Care. Cell Rep. 2017; 21 (11):3298-309.
16. Meacham C E, Morrison S J. Tumour heterogeneity and cancer cell plasticity. Nature. 2013; 501 (7467):328-37.
17. Poschke I C, Hassel J C, Rodriguez-Ehrenfried A, Lindner K A M, Heras-Murillo I, Appel L M, et al. The Outcome of Ex Vivo TIL Expansion Is Highly Influenced by Spatial Heterogeneity of the Tumor T-Cell Repertoire and Differences in Intrinsic In Vitro Growth Capacity between T-Cell Clones. Clin Cancer Res. 2020; 26 (16):4289-301.
18. Ren L, Matsuda T, Deng B, Kiyotani K, Kato T, Park J H, et al. Similarity and difference in tumor-infiltrating lymphocytes in original tumor tissues and those of in vitro expanded populations in head and neck cancer. Oncotarget. 2018; 9 (3):3805-14.
19. Langdon W B. Performance of genetic programming optimised Bowtie2 on genome comparison and analytic testing (GCAT) benchmarks. BioData Min. 2015; 8 (1): 1.
20. Nazarov V I, Pogorelyy M V, Komech E A, Zvyagin I V, Bolotin D A, Shugay M, et al. tcR: an R package for T cell receptor repertoire advanced data analysis. BMC Bioinformatics. 2015; 16:175.
21. Hosoi A, Takeda K, Nagaoka K, lino T, Matsushita H, Ueha S, et al. Increased diversity with reduced “diversity evenness” of tumor infiltrating T-cells for the successful cancer immunotherapy. Sci Rep. 2018; 8 (1): 1058.
22. Farmanbar A, Kneller R, Firouzi S. RNA sequencing identifies clonal structure of T-cell repertoires in patients with adult T-cell leukemia/lymphoma. NPJ Genom Med. 2019; 4:10.
23. Postow M A, Manuel M, Wong P, Yuan J, Dong Z, Liu C, et al. Peripheral T cell receptor diversity is associated with clinical outcomes following ipilimumab treatment in metastatic melanoma. J Immunother Cancer. 2015; 3:23.

### INCORPORATION BY REFERENCE

The entire disclosure of each of the patent documents, including certificates of correction, patent application documents, scientific articles, governmental reports, websites, and other references referred to herein is incorporated by reference herein in its entirety for all purposes. In case of a conflict in terminology, the present specification controls.

### EQUIVALENTS

The invention can be embodied in other specific forms without departing from the spirit or essential characteristics thereof. The foregoing embodiments are to be considered in all respects illustrative rather than limiting on the invention described herein. In the various embodiments of the systems, compositions and methods, where the term comprises is used with respect to the components of the systems or compositions or the recited steps of the methods, it is also contemplated that the systems, compositions, and methods consist essentially of, or consist of, the recited components or steps. Furthermore, it should be understood that the order of steps or order for performing certain actions is immaterial so long as the invention remains operable. Moreover, two or more steps or actions can be conducted simultaneously.

In the specification, the singular forms also include the plural forms, unless the context clearly dictates otherwise. Unless defined otherwise, all technical and scientific terms used herein have the same meaning as commonly understood by one of ordinary skill in the art to which this invention belongs. In the case of conflict, the present specification will control.

Furthermore, it should be recognized that in certain instances a composition can be described as being composed of the components prior to mixing, because upon mixing certain components can further react or be transformed into additional materials.

All percentages and ratios used herein, unless otherwise indicated, are by weight. It is recognized the mass of an object is often referred to as its weight in everyday usage and for most common scientific purposes, but that mass technically refers to the amount of matter of an object, whereas weight refers to the force experienced by an object due to gravity. Also, in common usage the “weight” (mass) of an object is what one determines when one “weighs” (masses) an object on a scale or balance.

What is claimed is:

1. A method for separation and analysis of biological particles utilizing a destructible hydrogel microwell comprising the steps of:
a) establishment of an array of hydrogel microwells,
b) seeding a sample of biological particles into the array,
c) culturing the sample, and
d) destruction of the microwells to release the cultured biological particles from the microwells.

2. The method according to claim 1, wherein the culturing step c) produces cultured biological particles within the microwells.

3. The method according to claim 1 comprising the further step, e) of quantitating and/or identifying the released particles from the destroyed microwells.

4. A method for separation and analysis of biological particles utilizing a destructible hydrogel microwell according to claim 1 comprising the further step x) between step c) and step d) of
x) segregation of targeted microwells from the array.

5. The method of claim 1 wherein the hydrogel is optically transparent.

6. The method of claim 5 wherein the hydrogel is transparent to light from about 315 nm to about 400 nm.

7. The method of claim 1 wherein each hydrogel microwell of the array has a diameter, width, or cross-sectional dimension from about 1 micron to 10 mm.

8. The method of claim 1 wherein the depth (inside height of the walls) of each hydrogel microwell of the array is from about 10 microns to about 500 microns.

9. The method of claim 1 wherein the volume of each hydrogel microwell of the array is from about 1×10⁻¹² liters to about 1×10⁻⁶ liters.

10. The method according to claim 1 wherein the array comprises from about 2 to about 1×10¹⁰ microwells.

11. The method according to claim 1 wherein the array comprises from about 1×10³ to about 1×10⁸ microwells.

12. The method of claim 1 wherein the microwell hydrogel array is a 2D array.

13. The method of claim 1 wherein the microwell hydrogel array is a 3D array.

14. The method of claim 1 wherein the hydrogel is selected from the group consisting of gelatin and its derivatives, agarose and its derivatives, dextran and its derivatives, cellulose and its derivatives, chitin and its derivatives, alginate and its derivatives, PEG and its derivatives, and combinations thereof.

15. The method of claim 1 wherein the hydrogel is established by a photo-initiator.

16. The method of claim 14 wherein the photo-initiator is lithium phenyl(2,4,6-trimethylbenzoyl)phosphinate.

17. The method of claim 1 where the hydrogel array is bound to or capable of adhering to a substrate.

18. The method of claim 17 wherein the substrate is selected from the group consisting of polystyrene, polyacrylate, polycarbonate, co-polymers of polystyrene, polyacrylate, and/or polycarbonate, and glass.

19. The method of claim 1 wherein the destruction of step d) is selected from the group consisting of partial destruction and complete destruction.

20. The method according to claim 1 wherein the destruction of step d) is performed by a method selected from the group consisting of,
i. chemical means (including enzymatic means),
ii. light means (including UV and visible),
iii. thermal means,
iv. sonic means (applying sound energy),
v. physical means,
vi. electromagnetic radiation,
vii. atomic particle means,
viii. subatomic particle means,
ix. biological means,
and combinations thereof.

21. The method of claim 1 wherein the destruction of step d) is performed by enzymatic digestion.

22. The method of claim 21 wherein the enzymatic digestion if performed with an enzyme selected from the group consisting of collagenase, trypsin, cellulose hydrolase, alginate lyase, dextranase, accutase, and combinations thereof.

23. The method of claim 21 wherein the enzymatic digestion is performed in the presence of EDTA or EGTA (also known as CAS 67-42-5 or ethyleneglycol-bis(β-aminoethyl)-N,N,N′,N′-tetraacetic Acid).

24. The method according to claim 1 wherein the biological particles are cells.

25. The method of claim 24 wherein the cells are selected from the group consisting of tumor cells, healthy cells, mutated cells, T-cells, lymphocytes, stem cells, circulating tumor cells, virus infected cells, adherent cells, suspension cells, and combinations thereof.

26. The method of claim 25 wherein the cells are selected from the group consisting of bacteria, plants, fungi, and combinations thereof.

27. The method of claim 26 wherein the cells are further carrying nucleic acid fragments, mutations in their genomes, plasmids, or wherein the cells are part of a microbiome containing a variety of microorganism species.

28. The method of claim 3 wherein the quantitation and/or qualitative analysis step e) is performed by morphology, kinetics, growth curve, cell killing, cell surface marker, migration, interaction, genome sequencing, fluorescence, illuminance, reporter gene expression, transcriptome sequencing, mass-spectrum, secreted proteins, and imaging.

29. A device (hardware and software) for generating a hydrogel microwell array of claim 1.

30. A destructible hydrogel microwell array construct of claim 1.

31. A method for preparing a destructible hydrogel microwell array of claim 1 comprising the steps of:
(a) depositing a polymerizable hydrogel onto a substrate, and
(b) initiating polymerization of the hydrogel with an energy source projected onto the hydrogel in a predetermined microarray arrangement to generate the microarray.

32. The method of claim 31 where the substrate is selected from the group consisting of polystyrene, polyacrylate, polycarbonate, co-polymers of polystyrene, polyacrylate, and/or polycarbonate, and glass.

33. The method of claim 32 wherein the deposition step (a) further comprises depositing a photo-initiator and a light absorption, and wherein in step (b) the energy source is a light source.

34. The method of claim 33 wherein the polymerizable hydrogel is selected from the group consisting of gelatin-methyl acrylate, dextran-methyl acrylate, and combinations thereof; the photoinitiator is lithium phenyl(2,4,6-trimethylbenzoyl)phosphinate; and the light absorption agent is tartrazine.

35. The method of claim 34 wherein the light source is a widefield (4 mm in diameter), narrow angle 405 nm laser.

36. The method of claim 35 wherein the laser is projected onto a 2K TFT (thin film transistor) monochrome LCD display to generate the microwell array.
